(19) United States
(12) Patent Application Publication
(10) Pub. No.: US 2022/0185754 A1
(43) Pub. Date: Jun. 16, 2022

(54) AN ALCOHOL RECOVERY SOLUTION, AND METHOD OF USE THEREFOR

(71) Applicant: Aquafortus Technologies Limited, Auckland (NZ)

(72) Inventors: Daryl Joseph BRIGGS, Auckland (NZ); Chaitra PRAKASH, Auckland (NZ)

(21) Appl. No.: 17/436,439

(22) PCT Filed: Mar. 6, 2020
(86) PCT No.: PCT/NZ2020/050019

§ 371 Date: Sep. 3, 2021

Related U.S. Application Data
(60) Provisional application No. 62/815,448, filed on Mar. 8, 2019.

(51) Int. Cl. C07C 29/86; B01D 11/04; C02F 1/26; C02F 1/58

(52) U.S. Cl. CPC C07C 29/86; B01D 11/0492; C02F 2101/34; C02F 1/58; B01D 2257/70; C02F 1/26

(57) ABSTRACT

The present invention is directed to a composition for use in recovering an alcohol from an aqueous solution the composition comprising: a) a recovery solution comprising at least one tertiary amine containing compound and at least one enolisable carbonyl; and, b) an aqueous process solution comprising the alcohol, wherein the recovery solution and the aqueous process solution are in direct contact, not miscible and at least a portion of the alcohol migrates from the aqueous process solution into the recovery solution.

### FIELD OF THE INVENTION

The present disclosure relates to an alcohol recovery solution. Also provided is a solution for use in a process that is suitable for separating or purifying alcohols, from an aqueous solution on a large scale and under energy efficient conditions.

### BACKGROUND OF THE INVENTION

Applicant has previously developed a thermoresponsive solution comprising a tertiary amine and enolisable carbonyl as published in WO2018/067019 and a salt recovery solution as published in WO/2019/070134.

Appropriate management of alcohol-containing waste streams can be challenging for manufacturers of consumer products, pharmaceuticals, industrial alcohols and beverages. Standard disposal options such as fuels blending and hazardous waste incineration are expensive and do not support reuse, recycle or waste minimization aspirations. Most solvent recovery plants rely on the distillation of the solvent from a waste stream and this is energy intensive. For example, many alcohol waste streams have very low concentrations of alcohol present in them and so there can be quite an energy commitment to recovery essentially small quantities of alcohol.

It is an object of the present invention to provide an alcohol recovery solution that overcomes these difficulties or to at least provide a useful alternative.

### SUMMARY OF THE INVENTION

The present invention in one aspect is directed to a composition for use in recovering an alcohol from an aqueous solution the composition comprising:
a) a recovery solution comprising at least one tertiary amine containing compound and at least one enolisable carbonyl; and,
b) an aqueous process solution comprising the alcohol,
wherein the recovery solution is not miscible with the aqueous process solution and at least a portion of the alcohol migrates from the aqueous process solution in the absence of a semipermeable membrane into the recovery solution.

In another aspect, the present invention provides a composition for use in recovering an alcohol from an aqueous solution the composition comprising:
a) a recovery solution comprising at least one tertiary amine containing compound and at least one enolisable carbonyl; and,
b) an aqueous process solution comprising the alcohol,
wherein the recovery solution and the aqueous process solution are in direct contact, not miscible and at least a portion of the alcohol migrates from the aqueous process solution into the recovery solution.

In another aspect, the present invention provides a composition for use in recovering an alcohol from an aqueous solution the composition comprising:
a) a recovery solution comprising at least one tertiary amine containing compound and at least one enolisable carbonyl; and,
b) an aqueous process solution comprising the alcohol,
wherein the recovery solution is not miscible with the aqueous solution.

In one embodiment the alcohol recovery solution comprises:
a) at least one tertiary amine containing compound; and
b) at least one enolisable carbonyl of Formula I,

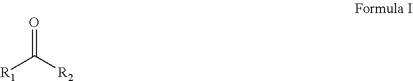

wherein
c) R₁ and R₂ are independently selected from a —C₁-C₂₀ alkyl and a —C₃-C₇ monocyclic; or
d) one of R₁ or R₂ is selected from a —O—(C₁-C₇ alkyl) and the other is selected from a —C₁-C₇ alkyl, or
e) R₁ and R₂ together, with the carbonyl of Formula I, form a 3-15 membered monocyclic ketone or a 3-15 membered monocyclic heterocyclic ketone or acetophenone.

In one embodiment, the alcohol in the aqueous process solution comprises alcohol selected from ethanol, butanol, isopropyl alcohol, methanol, tert-butanol or mixtures thereof.

In another aspect the present invention provides a method for separating an alcohol from an aqueous solution using a solvent recovery solution comprising at least one tertiary amine containing compound and at least one enolisable carbonyl, the method comprising the steps of:

bringing the alcohol containing aqueous solution into contact with the solvent recovery solution;
1) allowing one or more alcohols in the aqueous solution to migrate into the solvent recovery solution in the absence of a semipermeable membrane; and
2) separating the recovered alcohol from the aqueous solution.

In another aspect, the present invention is directed to the use of an alcohol recovery solution to recover an alcohol from an aqueous solution in the absence of a semipermeable membrane.

In one aspect the present invention provides the use of an alcohol recovery solution for use in recovering an alcohol from an aqueous solution in the absence of a semipermeable membrane, wherein the alcohol recovery solution comprises:
a) at least one tertiary amine containing compound; and
b) at least one enolisable carbonyl;
   wherein the alcohol recovery solution is not miscible with the aqueous solution.

The foregoing brief summary broadly describes the features and technical advantages of certain embodiments of the present invention. Further technical advantages will be described in the detailed description of the invention and examples that follows.

The foregoing brief summary broadly describes the features and technical advantages of certain embodiments of the present invention. Further technical advantages will be described in the detailed description of the invention and examples that follows.

Novel features that are believed to be characteristic of the invention will be better understood from the detailed description of the invention when considered in connection with any accompanying figures and examples. However, the figures and examples provided herein are intended to help illustrate the invention or assist with developing an understanding of the invention, and are not intended to limit the invention's scope.

### BRIEF DESCRIPTION OF THE DRAWINGS

FIG. 1: shows the osmolality of an aqueous solution containing ethanol in water plotted against the weight % of ethanol.

FIG. 2 shows the osmotic pressure of varying volume ratios of alcohol recovery solution with 5% ethanol in water at 40 degrees C.

FIG. 3 shows gas chromatogram traces of ethanol extraction using differing ratios of alcohol recovery solution.

FIG. 4 shows gas chromatogram traces of the alcohol recovery solution before and after exposure to the water (95%) to ethanol (5%) mix and the gas chromatogram traces of the water (95%) to ethanol (5%) mix before and after the exposure to the alcohol recovery solution.

FIG. 5 shows plots of osmotic pressure for standard solutions of various 2-nonanone, water, ethanol, tripentylamine solutions across a range of temperatures.

FIG. 6 shows plots of osmotic pressure for standard solutions of various 5-nonanone, water, ethanol, tripentylamine solutions across a range of temperatures.

FIG. 7 shows plots of osmotic pressure for standard solutions of various octanone, water, ethanol, tripentylamine solutions across a range of temperatures.

FIG. 8 shows a plot of ethanol absorption using different ratios of the alcohol recovery solution (Absorbent).

FIG. 9 shows a plot comparing the ethanol and water content in the different ratios of the alcohol recovery solution (Absorbent).

FIG. 10 shows a plot of the ethanol absorption and the water cross over into the alcohol recovery solution (Absorbent) at a ratio of 40:1, Absorbent:alcohol feed solutions having varying ethanol content (%).

FIG. 11 shows a plot of the ethanol absorption and the water cross over into the alcohol recovery solution (Absorbent) at a ratio of 40:1, Absorbent:alcohol feed solutions having varying ethanol content (%).

FIG. 12 shows a plot of butanol absorption using different ratios of the alcohol recovery solution (Absorbent).

FIG. 13 shows a plot comparing the butanol and water content in the different ratios of the alcohol recovery solution (Absorbent).

FIG. 14 shows a plot of isopropyl alcohol absorption using different ratios of the alcohol recovery solution (Absorbent).

FIG. 15 shows a plot comparing the butanol and water content in the different ratios of the alcohol recovery solution (Absorbent).

FIG. 16 shows a plot of the isopropyl absorption and the water cross over into the alcohol recovery solution (Absorbent) at a ratio of 40:1, Absorbent:alcohol feed solutions having varying isopropyl content (%).

FIG. 17 shows a plot of methanol absorption using different ratios of the alcohol recovery solution (Absorbent).

FIG. 18 shows a plot comparing the methanol and water content in the different ratios of the alcohol recovery solution (Absorbent)

FIG. 19 shows a plot of tert-butanol absorption using different ratios of the alcohol recovery solution (Absorbent).

FIG. 20 shows a plot comparing the tert-butanol and water content in the different ratios of the alcohol recovery solution (Absorbent).

FIG. 21 shows a plot showing how butan-1-ol can be concentrated through distillation.

### DETAILED DESCRIPTION OF THE INVENTION

The following description sets forth numerous exemplary configurations, parameters, and the like. It should be recognised, however, that such description is not intended as a limitation on the scope of the present invention but is instead provided as a description of exemplary embodiments.

### Definitions

In each instance herein, in descriptions, embodiments, and examples of the present invention, the terms “comprising”, “including”, etc., are to be read expansively, without limitation. Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

The term “about” or “approximately” usually means within 20%, more preferably within 10%, and most preferably still within 5% of a given value or range. Alternatively, the term “about” means within a log (i.e., an order of magnitude) preferably within a factor of two of a given value.

The term “immiscible” as used herein, means not fully miscible or capable of forming a single continuous phase with the solvent phase.

The term “water” as used throughout the specification means deionised water.

The term “semi-permeable” membrane as used herein, means a diffusion membrane such as a reverse osmosis membrane or a forward osmosis membrane or a nano-filtration membrane, and excludes a micro or ultra filtration membrane or size-based membrane.

As used herein, the term “C₁-C₂₀ alkyl” refers to a fully saturated branched or unbranched hydrocarbon moiety, which may be a straight or a branched chain of a particular range of 1-20 carbons. Preferably the alkyl comprises, 1 to 18, or 1 to 15, or 1 to 10, or 1 to 7 carbon atoms, or 1 to 4 carbon atoms. Representative examples of C₁-C₂₀ alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, n-dodecyl, n-pentadecyl, n-hexadecyl, n-heptadecyl, n-icosanyl and the like. For example, the expression C₁-C₄-alkyl includes, but is not limited to, methyl, ethyl, propyl, butyl, isopropyl, tert-butyl and isobutyl. In one embodiment the C₁-C₂₀ alkyl group may be substituted with one or more of the following groups: -halo, —OH, —CN, —NO₂, —C≡CH, —SH, —C₁-C₇ alkyl, —(C₁-C₇ alkyl)-OH, —NH₂, —NH(C₁-C₇ alkyl), —N(C₁-C₇ alkyl)₂, —O(C₁-C₇ alkyl), —C(O)—O(—C₁-C₇ alkyl), —C(O)OH; —C(O)—H, or —C(O)—(C₁-C₇ alkyl).

The term “C₃-C₇ monocyclic” as used herein is a 3-, 4-, 5-, 6-, or 7-membered saturated or unsaturated monocyclic ring. Representative C₃-C₇ monocyclic groups include, but are not limited to, cyclopropyl, cyclobutyl, cyclopentyl, cyclohexyl, phenyl, and cycloheptyl. In one embodiment, the C₃-C₇ monocyclic cycloalkyl group may be substituted with one or more of the following groups: -halo, —OH, —CN, —NO₂, —C≡CH, —SH, —C₁-C₇ alkyl, —(C₁-C₇ alkyl)-OH, —NH₂, —NH(C₁-C₇ alkyl), —N(C₁-C₇ alkyl)₂, —O(C₁-C₇ alkyl), —C(O)—O(—C₁-C₇ alkyl), —C(O)OH; —C(O)—H, or —C(O)—(C₁-C₇ alkyl).

The term “3- to 15-membered monocyclic ketone” refers to a 3- to 15-membered non-aromatic monocyclic ring system containing a ketone functional group. Representative examples of a 3- to 15-membered monocyclic ketone include, but are not limited to cyclopropanone, cyclobutanone, cyclopentanone, cyclohexanone, cycloheptanone, cyclooctanone, cyclononanone, cyclodecanone, cycloundecanone, cyclododecanone, cyclotridecanone; cyclotetradecanone and cyclopentadecanone.

In one embodiment, the 3- to 15-membered monocyclic ketone may be substituted with one or more of the following groups-halo, —OH, —CN, —NO₂, —C≡CH, —SH, —C₁-C₇ alkyl, —(C₁-C₇ alkyl)-OH, —NH₂, —NH(C₁-C₇ alkyl), —N(C₁-C₇ alkyl)₂, —O(C₁-C₇ alkyl), —C(O)—O(—C₁-C₇ alkyl), —C(O)OH; —C(O)—H, or —C(O)—(C₁-C₇ alkyl).

The term “3- to 15-membered monocyclic heterocyclic ketone” refers to: (i) a 3- or 4-membered non-aromatic monocyclic cycloalkyl in which 1 of the ring carbon atoms has been replaced with an N, O or S atom; or (ii) a 5- to 15-membered non-aromatic monocyclic cycloalkyl in which 1-4 of the ring carbon atoms have been independently replaced with a N, O or S atom. Representative examples of a 3- to 15-membered monocyclic heterocyclic ketone having one N, O or S atom include, but are not limited to oxiran-2-one, thiiran-2-one, oxetan-2-one, oxetan-3-one, azetidin-3-one, thietan-2-one, thietan-3-one, dihydrofuran-2(3H)-one, dihydrofuran-3(2H)-one, pyrrolidin-3-one, dihydrothiophen-3(2H)-one, dihydrothiophen-2(3H)-one, tetrahydro-2H-pyran-2-one, dihydro-2H-pyran-3(4H)-one, dihydro-2H-pyran-4(3H)-one, piperidin-3-one, piperidin-4-one, tetrahydro-2H-thiopyran-2-one, dihydro-2H-thiopyran-3(4H)-one, dihydro-2H-thiopyran-4(3H)-one, oxepan-2-one, oxepan-3-one, oxepan-4-one, thiepan-2-one, thiepan-3-one, thiepan-4-one, azepan-3-one, azepan-4-one, oxocan-2-one, oxocan-3-one, oxocan-4-one, oxocan-5-one, thiocan-2-one, thiocan-3-one, thiocan-4-one, thiocan-5-one, azocan-3-one, azocan-3-one, azocan-4-one, azocan-5-one, azonan-3-one, azonan-4-one, azonan-S-one, oxonan-2-one, oxonan-3-one, oxonan-4-one, oxonan-5-one, thionan-2-one, thionan-3-one, thionan-4-one, thionan-5-one, oxacycloundecan-2-one, oxacycloundecan-3-one, oxacycloundecan-4-one, oxacycloundecan-5-one, oxacycloundecan-6-one, azacycloundecan-3-one, azacycloundecan-4-one, azacycloundecan-5-one, azacycloundecan-6-one, thiacycloundecan-2-one, thiacycloundecan-3-one, thiacycloundecan-4-one, thiacycloundecan-5-one, thiacycloundecan-6-one, oxacyclododecan-2-one, oxacyclododecan-3-one, oxacyclododecan-4-one, oxacyclododecan-5-one, oxacyclododecan-6-one, oxacyclododecan-7-one, azacyclododecan-3-one, azacyclododecan-4-one, azacyclododecan-5-one, azacyclododecan-6-one, azacyclododecan-7-one, thiacyclododecan-2-one, thiacyclododecan-3-one, thiacyclododecan-4-one, thiacyclododecan-5-one, thiacyclododecan-6-one, thiacyclododecan-7-one, oxacyclotridecan-2-one, oxacyclotridecan-3-one, oxacyclotridecan-4-one, oxacyclotridecan-5-one, oxacyclotridecan-6-one, oxacyclotridecan-7-one, azacyclotridecan-3-one, azacyclotridecan-4-one, azacyclotridecan-5-one, azacyclotridecan-6-one, azacyclotridecan-7-one, thiacyclotridecan-2-one, thiacyclotridecan-3-one, thiacyclotridecan-4-one, thiacyclotridecan-5-one, thiacyclotridecan-6-one, thiacyclotridecan-7-one, oxacyclotetradecan-2-one, oxacyclotetradecan-3-one, oxacyclotetradecan-4-one, oxacyclotetradecan-5-one, oxacyclotetradecan-6-one, oxacyclotetradecan-7-one, oxacyclotetradecan-8-one, azacyclotetradecan-3-one, azacyclotetradecan-4-one, azacyclotetradecan-5-one, azacyclotetradecan-6-one, azacyclotetradecan-7-one, azacyclotetradecan-8-one, thiacyclotetradecan-2-one, thiacyclotetradecan-3-one, thiacyclotetradecan-4-one, thiacyclotetradecan-5-one, thiacyclotetradecan-6-one, thiacyclotetradecan-7-one, thiacyclotetradecan-8-one, oxacyclopentadecan-2-one, oxacyclopentadecan-3-one, oxacyclopentadecan-4-one, oxacyclopentadecan-5-one, oxacyclopentadecan-6-one, oxacyclopentadecan-7-one, oxacyclopentadecan-8-one, azacyclopentadecan-3-one, azacyclopentadecan-4-one, azacyclopentadecan-5-one, azacyclopentadecan-6-one, azacyclopentadecan-7-one, azacyclopentadecan-8-one, thiacyclopentadecan-2-one, thiacyclopentadecan-3-one, thiacyclopentadecan-4-one, thiacyclopentadecan-5-one, thiacyclopentadecan-6-one, thiacyclopentadecan-7-one, thiacyclopentadecan-8-one. In one embodiment, the 3- to 15-membered monocyclic heterocyclic ketone group may be substituted with one or more of the following groups-halo, —OH, —CN, —NO₂, —C≡CH, —SH, —C₁-C₆ lower alkyl, —(C₁-C₇ alkyl)-OH, —NH₂, —NH(C₁-C₇ alkyl), —N(C₁-C₇alkyl)₂, —O(C₁-C₇alkyl), —C(O)—O(—C₁-C₇alkyl), —C(O)OH; —C(O)—H, or —C(O)—(C₁-C₇ alkyl). For the avoidance of doubt, the 3-5 membered monocyclic heterocyclic ketone does not include any amide groups where the ketone enolisable carbonyl group is adjacent a N atom in the cyclic structure.

The term “halo” as used herein refers to —F, —Cl, —Br or —I.

The term “an enolisable carbonyl” means a compound that has one or more carbonyl functional groups and wherein at least one of the carbonyl functional groups has alpha hydrogens (Hα) that may be removed by a base to form an enolate and then an enol as shown in the reaction scheme below.

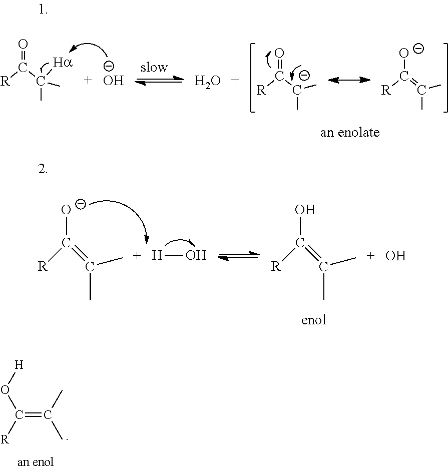

The term enolisable carbonyl, without limitation includes 2-octanone and 5-nonaone. It is to be understood that the term enolisable carbonyl as used in the specification does not include a compound having solely an aldehyde functional group, a compound having solely a carboxylic acid functional group, a compound having solely an amide functional group, a compound having solely an acyl halide functional group or acetylacetone.

The term “tertiary amine containing compound” preferably means a compound having at least one tertiary amine group, but it is to be appreciated that the compound may have more than one tertiary amine group or further may be a mixture of tertiary amine containing compounds. Preferably the tertiary amine containing compound is a base, such as a Lewis base. If the base is a lewis base, it is envisaged that a lewis adduct may be formed with the enolisable carbonyl. The solution may include a combination of more than one tertiary amine containing compound. The tertiary amine containing compound may be aliphatic, conjugated, asymmetric or cyclic or a combination thereof.

Examples of suitable tertiary amine containing compounds include, but are not limited to the following:

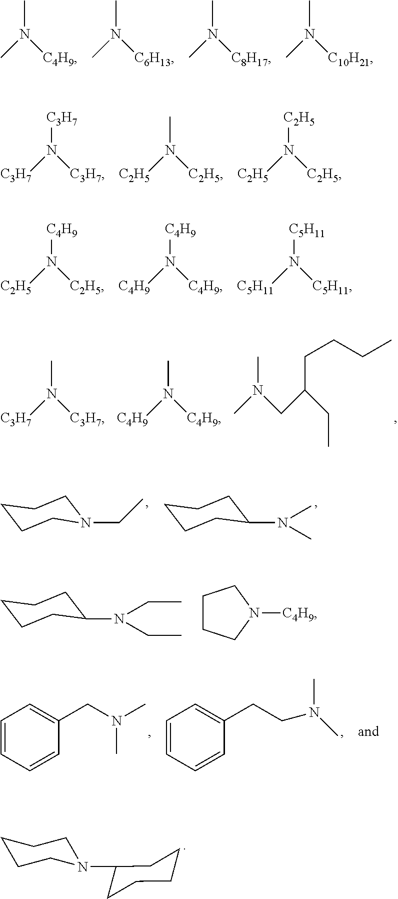

In one embodiment the tertiary amine containing compound is selected from a —N(C₁-C₇ alkyl)₃. In yet a further embodiment the tertiary amine containing compound is —N(C₅ alkyl)₃ (tripentylamine) or —N(C₄alkyl)₃ tributylamine.

It will be appreciated that the above listed tertiary amine containing compounds are simple enough for production on an industrial scale.

The present invention is directed to an alcohol recovery solution and its use to recover an alcohol from an aqueous solution. The inventors have conducted research into looking for alcohol solutions that are likely to be readily scalable on an industrial scale, whilst also providing very efficient diffusion and osmotic potential properties both cost and energy efficiently. The inventors have determined that a suitable alcohol recovery solution comprises:
a) at least one tertiary amine containing compound; and
b) at least one enolisable carbonyl of Formula I,

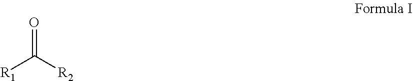

wherein
c) R₁ and R₂ are independently selected from a —C₁-C₂₀ alkyl or a —C₃-C₇ monocyclic; or
d) one of R₁ or R₂ is selected from a —O—(C₁-C₇ alkyl) and the other is selected from a —C₁-C₂₀ alkyl, or
e) R₁ and R₂ together, with the carbonyl of Formula I, form a 3-15 membered monocyclic ketone or a 3-15 membered monocyclic heterocyclic ketone; and
   wherein in use the alcohol recovery solution is not miscible with an aqueous solution from which the alcohol is to be recovered.

In one embodiment, R₁ and R₂ of Formula I are independently selected from a —C₁-C₂₀ alkyl. In another embodiment R₁ and R₂ are independently selected from methyl and ethyl. In one embodiment the enolisable carbonyl is selected from 2-butanone, acetone, isobutylketone, 5-nonanone, 2-octanone. In one embodiment the solution includes a combination of more than one enolisable carbonyl of Formula I.

In one embodiment R₁ of Formula I is selected from a —C₁-C₂₀ alkyl and R₂ is selected from a —O—(C₁-C₇ alkyl). In a further embodiment the enolisable carbonyl is selected from ethyl formate or methyl formate.

In a further embodiment wherein R₁ and R₂ of Formula I together form a cyclic system selected from a 3-15 membered monocyclic ketone or a monocyclic ester. In one embodiment the enolisable carbonyl is selected from 2-octanone.

It is to be appreciated that when R₁ and R₂ together, with the carbonyl group of Formula I, form a cyclic system, the cyclic system may be further substituted with one or more substituents selected from -halo, —OH, —CN, —NO₂, —C≡CH, —SH, —C₁-C₇ alkyl, —(C₁-C₇ alkyl)-OH, —NH₂, —NH(C₁-C₇ alkyl), —N(C₁-C₇ alkyl)₂, —O(C₁-C₇ alkyl), —C(O)—O(—C₁-C₇ alkyl), —C(O)OH; —C(O)—H, or —C(O)—(C₁-C₇ alkyl) or the like.

It is to be appreciated that the molar ratio of the tertiary amine containing compound to the enolisable carbonyl of Formula I may vary widely and may be from about 1:99 or 99:1; or from about 1:50 or 50:1 or from about 1:10 or 10:1 or from about 1:5 or 5:1 or from about 1:3 or from about 3:1 or from about 1:2 or from about 2:1. In a preferred embodiment the molar ratio is about 1:1. It would be readily apparent to an industrial chemist how to determine the most suitable molar ratio to be employed.

It is to be appreciated that the alcohol recovery percentage may vary widely, however, preferred alcohol recovery rates range from 5 to 100%, more preferably from 10 to 100%, more preferably from 15 to 100%. The alcohol recovery percentage will also depend on how many times the alcohol in the aqueous solution is exposed to the alcohol recovery solution, such as 1 passage or multiple passages through an alcohol recovery solution.

In a further aspect there is provided a method for separating an alcohol from an aqueous solution using an alcohol recovery solution as defined above, the method comprising the steps of:
1) bringing the alcohol containing aqueous solution into contact with the alcohol recovery solution;
2) allowing one or more alcohol solvents in the aqueous solution to at least partially migrate into the alcohol recovery solution where a recovered alcohol containing emulsion is formed; and
3) separating the recovered alcohol containing emulsion from the aqueous solution.

It is to be appreciated that the aqueous solution may be selected from industrial water waste streams, fermentation streams, food & beverage processing effluents, or the like.

It is to be appreciated that the molar ratio of alcohol to alcohol recovery solution as defined above may vary widely and may be from about 1:1 to about 1:100, or from about 1:1 to about 1:50 or from about 1:1 to about 1:20.

### EXAMPLES

The examples described herein are provided for the purpose of illustrating specific embodiments of the invention and are not intended to limit the invention in any way. Persons of ordinary skill can utilise the disclosures and teachings herein to produce other embodiments and variations without undue experimentation. All such embodiments and variations are considered to be part of this invention.

### Example 1

Alcohol recovery solutions were tested to separate the solution containing 5% ethanol in 95% water. Because the alcohol recovery solution remained bi-phasic throughout the testing and was hydrophobic in nature, fractionating or absorbing ethanol from water or an aqueous solution using the alcohol recovery solution, which is not soluble in water is a possibility. The following experimental work was conducted.

All the tests in this experiment were performed at a single temperature −40° C. The presence of ethanol in water or in the organic layer was tested using an Osmomat-3000 instrument which gives the osmolality data of the aqueous layer of the various solutions.

Osmolality data was also obtained for the alcohol recovery solution containing an entrainer (in this case, cyclohexane). Entrainers are introduced in azeotropic distillation processes to form an azeotrope with ethanol at lower temperatures than water.

### Reference: Osmolality Data of Ethanol in Water

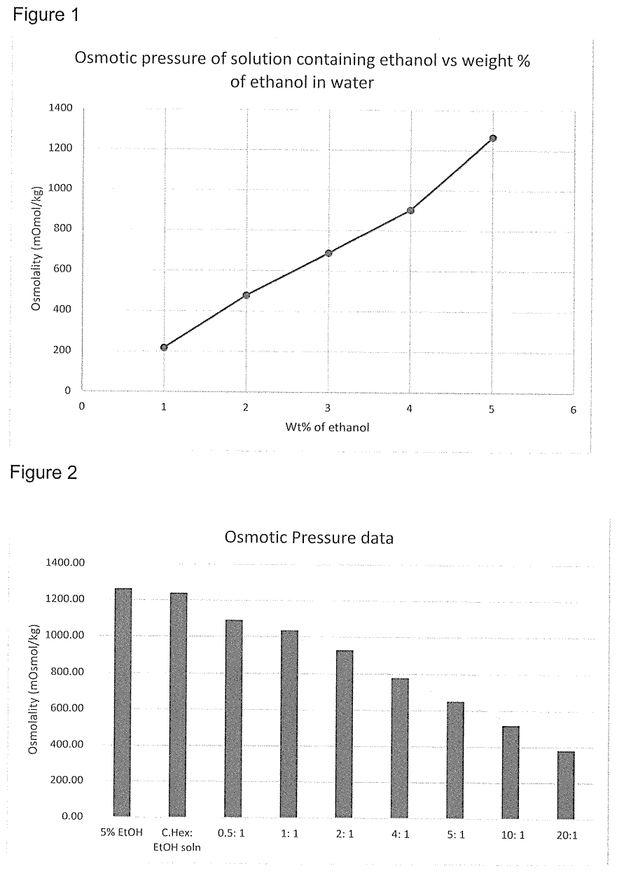

Samples were prepared with test solutions containing ethanol and water. The weight percentages of ethanol varied from 1 to 5 in water. 50 μL of the test solution was pipetted out into sample holders of the Osmometer and the sample measurement was performed automatically, and the osmolality was displayed on the screen. Three measurements were taken for each sample and the resulting value was averaged as shown in Table 1 and FIG. 1.

TABLE 1

| Weight % of | Weight % of | Osmolality (mOsmol/kg) | | | |
|---|---|---|---|---|---|
| ethanol | water | Trial 1 | Trial 2 | Trial 3 | Average |
| 1 | 99 | 218 | 215 | 218 | 217 |
| 2 | 98 | 479 | 476 | 472 | 475.67 |
| 3 | 97 | 685 | 688 | 684 | 685.67 |
| 4 | 96 | 905 | 893 | 904 | 900.67 |
| 5 | 95 | 1268 | 1274 | 1252 | 1264.67 |
| 5% at 40° C. | 95 | 1248 | 1267 | 1267 | 1260.67 |

### Example 2 Tripentylamine: 2-Octanone (0.5:1 Molar Ratio)

Standards solutions were prepared as follows:
5 mL Tripentylamine and 5 mL DI water
5 mL 2-Octanone and 5 mL DI water
5% Ethanol and 95% water (10 mL solution)
Cyclohexane (5 mL): (5% ethanol in 95% water) —5 mL—1:1 by volume
2-Octanone (5 mL): (5% ethanol in 95% water) (5 mL) —1:1 by volume
Tripentylamine (5 mL): (5% ethanol in 95% water) (5 mL) —1:1 by volume

The osmolality of the aqueous layer of the standard solutions was recorded on a Knauer semi-micro Osmometer K-7400 over a range of temperatures (20° C., 40° C., 60° C., 80° C. and 100° C.) and compared to that of the values obtained from the alcohol recovery solution containing ethanol. The temperature of the solvent recovery solution was regulated using a temperature controller (Qpod 2e) with constant stirring at 1500 rpm. 50 μL of aqueous phase of the sample (each standard solution) was pipetted out into the measuring vessel and attached to the thermistor probe of the Osmometer. The sample measurement was performed automatically, and the osmolality was displayed on the screen.

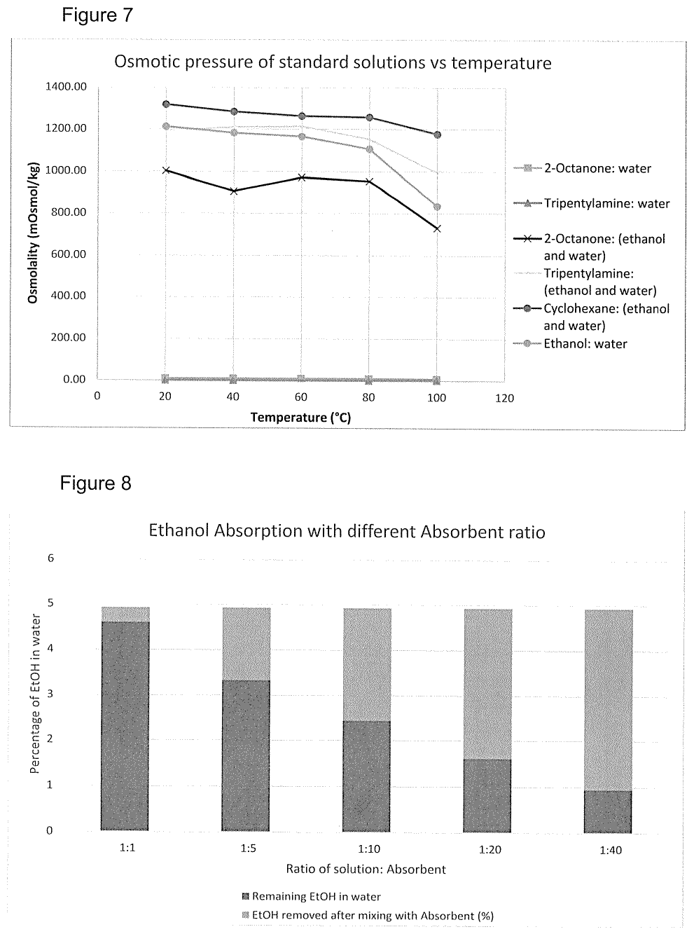

The plot of osmotic pressure of all the standard solutions vs temperature is shown in FIG. 7. From FIG. 7, it is possible to conclude that there was no presence of the amine or ketone in the aqueous layer because the osmolality measurements corresponded to close to zero. In contrast, in the presence of ethanol, the osmotic pressure of the aqueous layer increased indicating that it is not being absorbed by the individual components of the draw solution. There was no significant osmolality changes seen with increased temperatures.

### Tripentylamine: 2-Octanone (0.5:1 Molar Ratio)

The osmolality of the aqueous phase of the alcohol recovered solution with 5% of ethanol (by weight) was determined at 40° C. The ratio of the pure solvent (in this case alcohol) recovery solution was varied from 0.5 to 20 and the resulting osmotic pressure of the aqueous phase was determined. The temperature of the solvent recovery solution was regulated using a temperature controller (Qpod 2e) with constant stirring at 1500 rpm. 50 μL of aqueous phase of the sample was pipetted out into the measuring vessel and attached to the thermistor probe of the Osmometer. The sample measurement was performed automatically, and the osmolality was displayed on the screen. Three measurements were taken for each sample and the resulting value was averaged as shown in Table 2 and FIG. 2.

TABLE 2

| Volume ratio of | | | | |
|---|---|---|---|---|
| alcohol recovery | Osmolality (mOsmol/kg) | | | |
| solution:water | Measure- | Measure- | Measure- | |
| with 5% ethanol | ment 1 | ment 2 | ment 3 | Average |
| 0.5:1 | 1082 | 1115 | 1074 | 1090.33 |
| 1:1 | 1051 | 1023 | 1028 | 1034 |
| 2:1 | 934 | 917 | 937 | 929.33 |
| 4:1 | 781 | 774 | 780 | 778.33 |
| 5:1 | 658 | 642 | 650 | 650 |
| 10:1 | 521 | 519 | 520 | 520 |
| 20:1 | 382 | 380 | 382 | 381.33 |

The osmotic pressure data of the solutions with varying volume ratio of the recovery solution was also plotted as a histogram. The results are shown in FIG. 2 in histogram format.

From the above results, it can be seen that the ethanol absorption is related to the volume ratio of the recovery solution. The osmolality of the water decreased with the increase in the volume ratio of the recovery solution. The liquid-liquid partitioning effect is observed as the ratio of the recovery solution increased from 0.5 to 20.

### Example 3 Tripentylamine (TPA): 2-nonanone 2-NONE (0.5:1 Molar Ratio)

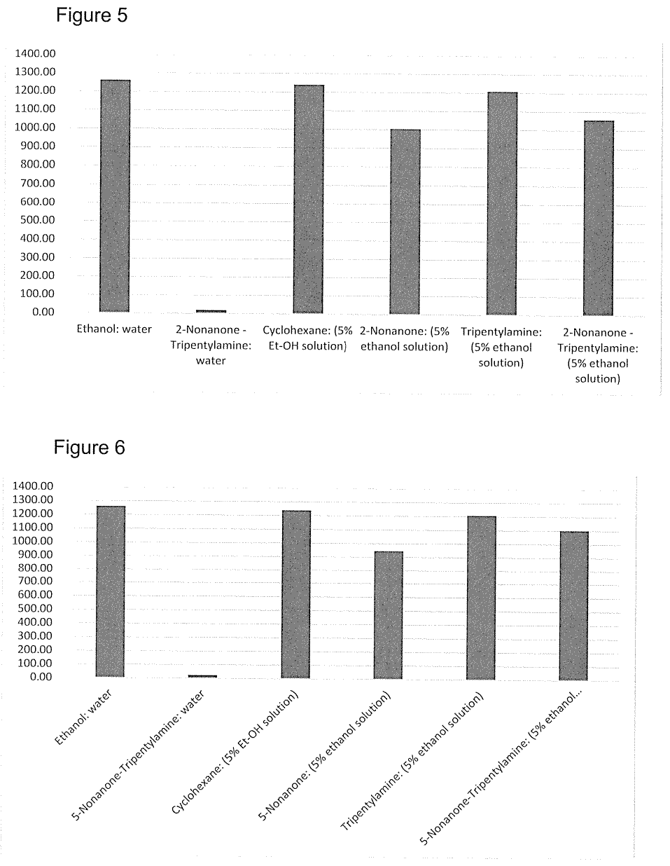

The osmolality of the aqueous phase of the alcohol recovered solution with 5% of ethanol (by weight) was determined at 40° C. The ratio of the pure solvent (in this case alcohol) recovery solution was 0.5:1 and the resulting osmotic pressure of the aqueous phase was determined. The temperature of the solvent recovery solution was regulated using a temperature controller (Qpod 2e) with constant stirring at 1500 rpm. 50 μL of aqueous phase of the sample was pipetted out into the measuring vessel and attached to the thermistor probe of the Osmometer. The sample measurement was performed automatically, and the osmolality was displayed on the screen. Three measurements were taken for each sample and the resulting value was averaged as shown in Tables 3 and 4 and FIG. 5.

TABLE 3

| | Osmolality (mOsmol/kg) | | | |
|---|---|---|---|---|
| | Measure- | Measure- | Measure- | |
| | ment 1 | ment 2 | ment 3 | Average |
| Alcohol recovery solution | 18 | 18 | 18 | 18 |
| (2-NONE:TPA):water | | | | |
| 2-NONE:5% Ethanol | 1010 | 1011 | 990 | 1003.67 |
| solution | | | | |
| Alcohol recovery solution | 1079 | 1043 | 1051 | 1057.67 |
| (2-NONE:TPA):5% | | | | |
| Ethanol solution | | | | |

TABLE 4

| | Osmolality of the solutions (mOsmol/kg) | | | | | |
|---|---|---|---|---|---|---|
| | | | Cyclohexane:(5% | 2-NONE:(5% | TPA:(5% | 2-NONE-TPA:(5% |
| | | | EtOH | ethanol | ethanol | ethanol |
| Temp (° C.) | Ethanol:water | 2-NONE:TPA:water | solution) | solution) | solution) | solution) |
| 40 | 1260.00 | 18 | 1238.00 | 1003.67 | 1208.00 | 1057.67 |

The osmolality of the solutions shown in Table 4 are also shown plotted in FIG. 5.

### Example 4: Tripentylamine (TPA): 5-nonanone 5-NONE (0.5:1 Molar Ratio)

The osmolality of the aqueous phase of the alcohol recovery solution with 5% of ethanol (by weight) was determined at 40° C. The ratio of the pure solvent (in this case alcohol) recovery solution was 0.5:1 and the resulting osmotic pressure of the aqueous phase was determined. The temperature of the solvent recovery solution was regulated using a temperature controller (Qpod 2e) with constant stirring at 1500 rpm. 50 μL of aqueous phase of the sample was pipetted out into the measuring vessel and attached to the thermistor probe of the Osmometer. The sample measurement was performed automatically, and the osmolality was displayed on the screen. Three measurements were taken for each sample and the resulting value was averaged as shown in Tables 5 and 6 and FIG. 6.

TABLE 5

| | Osmolality (mOsmol/kg) | | | |
|---|---|---|---|---|
| | Measure- | Measure- | Measure- | |
| | ment 1 | ment 2 | ment 3 | Average |
| Alcohol recovery solution | 23 | 24 | 24 | 23.67 |
| (5-NONE:TPA):water | | | | |
| 5-NONE:5% Ethanol | 934 | 949 | 950 | 944.33 |
| solution | | | | |
| Alcohol recovery solution | 1090 | 1109 | 1106 | 1101.67 |
| (5-NONE:TPA):5% | | | | |
| Ethanol solution | | | | |

TABLE 6

| | Osmolality of the solutions (mOsmol/kg) | | | | | |
|---|---|---|---|---|---|---|
| | | | Cyclohexane: | 5-NONE:(5% | TPA:(5% | 5-NONE-TPA: |
| | Ethanol: | 5-NONE: | (5% EtOH | ethanol | ethanol | (5% ethanol |
| Temp (° C.) | water | TPA:water | solution) | solution) | solution) | solution) |
| 40 | 1260.00 | 23.67 | 1238.00 | 944.33 | 1208.00 | 1101.67 |

The osmolality of the solutions shown in Table 6 are also shown plotted in FIG. 6.

### Example 5: Ethanol Extraction Examples

The ability of an alcohol recovery solution to recover ethanol was assessed under a number of different conditions as described below.

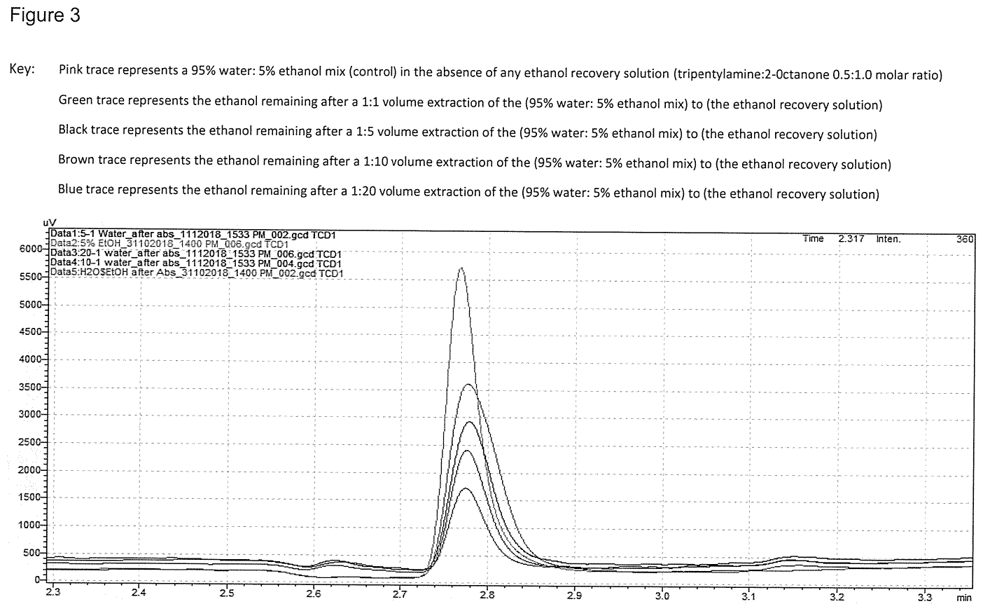

In a first example, a 95% water to 5% ethanol mixture was added to an alcohol recovery solution comprising tripentylamine and 2-octanone in a 0.5:1 molar ratio on a varying volume to volume basis. Gas chromatogram analysis of the resulting solution was obtained, and the results are tabulated below in Table 7. The results are also shown in FIG. 3.

TABLE 7
Showing the residual ethanol after extraction and ethanol recovery.

| Water/ethanol mix: | | | | |
|---|---|---|---|---|
| alcohol recovery | Peak | | | |
| solution | position | Peak Area | % Ethanol | % Recovery |
| 1:0 (control) | 2.767 | 16267 | 5.0 | — |
| 1:1 | 2.777 | 14525 | 4.5 | 10.7 |
| 1:5 | 2.778 | 9382 | 2.9 | 42.3 |
| 1:10 | 2.776 | 7113 | 2.2 | 56.3 |
| 1:20 | 2.775 | 4493 | 1.4 | 72.4 |

Whilst the recovery rate depends on the ratio of the alcohol recovery solution present, significant % recovery of ethanol can be achieved.

All GC data for Example 5 was collected on a SHIMADZU Nexis 2030 gas chromatograph fitted with a SUPELCO WATERCOL 1910 column. The GC parameters were set up as shown below:

| Parameter | Setting | |
|---|---|---|
| Injection Volume | 1.0 | μL |
| Injection temperature | 250° | C. |
| Injection mode | Split | |
| Split ratio | 100.0 | |
| Carrier gas | He | |
| Carrier gas pressure | 53.1 | kPa |
| Column flow | 0.93 | mL/min |
| Liner velocity | 22.0 | cm/s |
| Column length | 30.0 | m |
| Column inner diameter | 0.32 | |
| Column method | Isocratic | |
| Column temperature | 163.0° | C. |
| Total time | 9 | min |
| Detector | TCD | |
| TCD sample rate | 40 | ms |
| TCD current | 70 | mA |
| Makeup gas | He | |
| Makeup flow | 8.0 | mL/min |
| TCD temperature | 200° | C. |

### Column Method:

| Rate (° C./min) | Temperature (° C.) | Hold Time (min) |
|---|---|---|
| | 100.00 | 2.55 |
| 25.0 | 168.0 | 5.0 |

Total program time 10:27 min

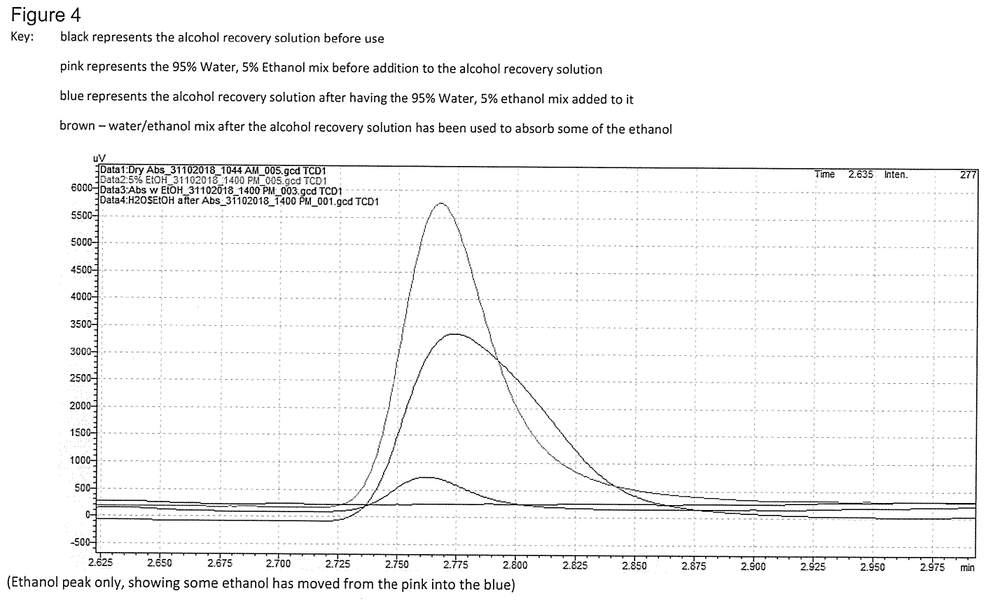

In a further example, a 95% water to 5% ethanol mixture was added to an alcohol recovery solution comprising tributylamine and 5-nonanone in a 0.5:1 molar ratio on a varying volume to volume basis. Gas chromatogram analysis of the resulting solution was obtained, and the results are shown in FIG. 4.

It is to be appreciated that from these results, it is apparent that a waste stream containing as little as 5% ethanol by weight in for example 95% water could be utilized in an ethanol recovery process as described above. The ethanol could be recovered by bringing the waste stream into contact with an alcohol recovery solution as described herein. The ethanol would be largely absorbed into the solvent recovery solution for extraction therefrom.

### Example 6 Quantification of Ethanol that Migrates into the Alcohol Recovery Solution

All GC data for Examples 6 to 9 was collected on a SHIMADZU Nexis 2030 gas chromatograph fitted with a SUPELCO WATERCOL 1910 column. The GC parameters were set up as shown below:

| Parameter | Setting | |
|---|---|---|
| Injection Volume | 0.5 | μL |
| Injection temperature | 250° | C. |
| Injection mode | Split | |
| Split ratio | 50.0 | |
| Carrier gas | He | |
| Carrier gas pressure | 53.1 | kPa |
| Column | SH-Rxi-624Sil MS | |
| Column flow | 1.16 | mL/min |
| Liner velocity | 24.0 | cm/s |
| Column length | 30.0 | m |
| Column inner diameter | 0.32 | |
| Column method | Gradient | |
| Column temperature | 163.0° | C. |
| Total time | 16 | min |
| Detector | TCD | |
| TCD sample rate | 40 | ms |
| TCD current | 60 | mA |
| Makeup gas | He | |
| Makeup flow | 8.0 | mL/min |
| TCD temperature | 200° | C. |

### Column Method:

| Rate (° C./min) | Temperature (° C.) | Hold Time (min) |
|---|---|---|
| | 100.00 | 2.00 |
| 15.0 | 250 | 4.00 |

Total program time 16.00 min

A solution of 5% EtOH in water (2 ml of EtOH in 38 ml of water) was prepared. An alcohol recovery solution comprising a 0.5:1 mole ratio of tripentylamine and 2-octanone was prepared. Gas chromatography calibration curves (using GC parameters defined above) of EtOH and water and tripentylamine, 2-octanone and water and tripentylamine, 2-Octanone and EtOH were obtained.

1 ml of 5% EtOH aqueous feed solution was added to 20 ml of the alcohol recovery solution of tripentylamine and 2-octanone. The resulting mixture was mixed by vortex and then centrifuged to separate out the respective layers. The alcohol recovery solution (top phase) and the mixture (bottom phase) were analysed by GC (using GC parameters defined above).

### Results

The following results tabulated in Table 8 were obtained by GC analysis.

TABLE 8

| | | | % of ethanol in 20 ml of |
|---|---|---|---|
| | % of ethanol content | % of ethanol left in aqueous | recovery solution after |
| Original ethanol | actually measured by | feed solution after coming | coming into direct |
| content in aqueous | GC in aqueous feed | into direct contact with | contact with feed |
| feed solution % | solution | alcohol recovery solution | solution |
| 5% (1 ml) | 4.921% (or 0.04921 ml | 1.73%* | 0.168%^{#} |
| | per 1 ml of solution) | | |

*This means that (4.921%-1.73%) = 3.191% of EtOH in the aqueous feed solution was removed by the alcohol recovery solution or that 0.03191 ml of EtOH was removed per 1 ml of solution.
^{#}The EtOH in the alcohol recovery solution is 0.168%, which means that 0.0336 ml of EtOH was present in the 20 ml of absorbent.

Some water 0.312% (0.0624 ml) was also measured to have passed into the 20 ml of alcohol recovery solution.

### Calculations

EtOH recovery=Volume of EtOh in Alcohol recovery solution/Volume of EtOH in water*100%=0.0336/0.04921*100%=68.28%

Water crossover=Volume of water in Alcohol recovery solution/Volume of water added*100%=0.0624/1*100%=6.24%

In conclusion, it can be sees that the alcohol recovery solution has extracted the EtOH (0.0336 ml) from the EtOH/H2O mixture, which equates to a 68% recovery of EtOH. It must be noted, however, that some water (0.0624 ml) also migrated into the alcohol recovery solution. Overall in the example shown above, the EtOH recovery is 68.28% and the water crossover is 6.24%.

### Example 7: Tripentylamine (TPA): 2-Octanone (0.5:1 Molar Ratio) Used for Ethanol Recovery

A 5% ethanol (2 mls) in water (in 38 mls of water) solution was prepared and analysed by GC.

The alcohol recovery solution (also referred to as the alcohol absorbent solution) was prepared as a 0.5:1 mole ratio of tripentylamine and 2-octanone by combining 289 ml of tripentylamine to 311 ml of 2-octanone and mixing by shaking for 10 secs.

Gas chromatography calibration curves of ethanol with water and tripentylamine, 2-octanone with water and tripentylamine, 2-octanone and ethanol were obtained by per the GC parameters outlined in Example 6.

1 ml of the 5% ethanol solution was added to various ratios (1:1, 1:5, 1:10, 1:20 and 1:40) of the tripentylamine and 2-octanone alcohol recovery solution to prepare ratios as follows:
A. 1:1 ratio was prepared by mixing 1 ml of 5% EtOH aqueous feed solution with 1 ml of the alcohol recovery solution of tripentylamine and 2-octanone;
B. 1:5 ratio was prepared by mixing 1 ml of 5% EtOH aqueous feed solution with 5 ml of the alcohol recovery solution of tripentylamine and 2-octanone;
C. 1:10 ratio was prepared by mixing 1 ml of 5% EtoH aqueous feed solution with 10 ml of the alcohol recovery solution of tripentylamine and 2-octanone;
D. 1:20 ratio was prepared by mixing 1 ml of 5% EtoH aqueous feed solution with 20 ml of the alcohol recovery solution of tripentylamine and 2-octanone;
E. 1:40 ratio was prepared by mixing 1 ml of 5% EtoH aqueous feed solution with 40 ml of the alcohol recovery solution of tripentylamine and 2-octanone.

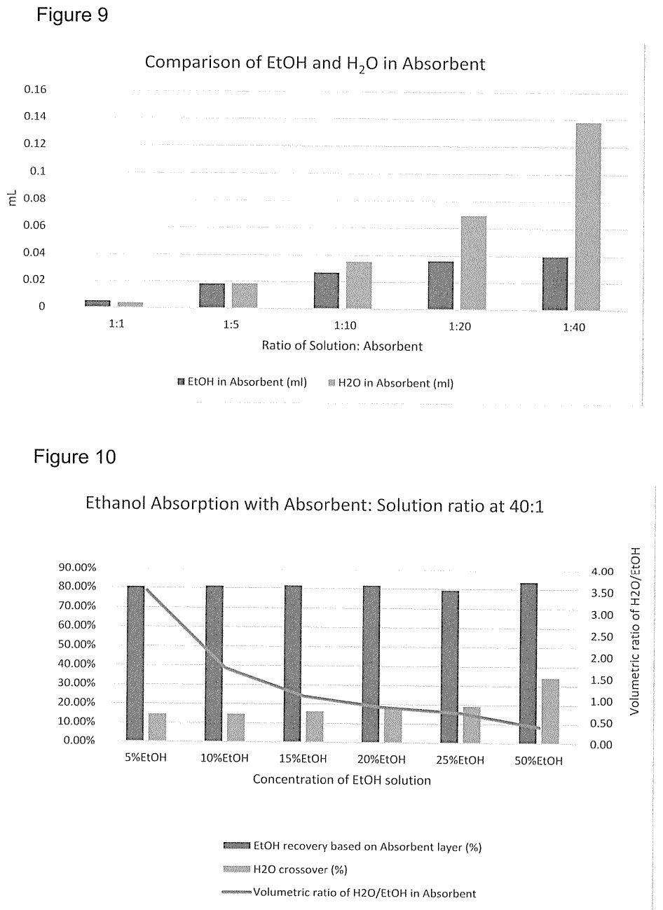

The resulting mixtures were vortexed for 30 secs and then centrifuged for 1 min at 400 rpm to separate into two layers. The alcohol recovery solution of tripentylamine and 2-octanone (or alcohol absorbent solution “Absorbent”) was the top phase and the bottom phase was the ethanol solution in water. The composition of the top phase and the bottom phase were analysed by GC per the parameters outlined in Example 6 above to determine a number of parameters, including the percentage of ethanol in water after mixing with the Absorbent, the ethanol remaining in the water after mixing with the Absorbent. These parameters were then used to calculate the ethanol extracted from the water, the percentage of the total ethanol removed, the mls and percentage of ethanol in the Absorbent, the mls and percentage of water in the Absorbent and the percentage of water crossover into the Absorbent. The alcohol/ethanol recovery and water crossover was calculated using the equations below and the results are shown in Tables 9(a) and 9(b) and in FIGS. 8 and 9.

$$Alcohol recovery = \frac{Volume of alcohol obtained}{Volumne of alcohol in water} \times 100 \%$$

$$Water crossover = \frac{Volumne of water in Absorbent}{Volumne of water added} \times 100 \%$$

$${K}_{a w} = \frac{Percentage of EtOH in Absorbent}{Percentage of EtOH removed from water} \times 100 \%$$

TABLE 9(a)
Ethanol recovery with different volume of Water/Ethanol to Absorbent ratio

| | | EtOH in | EtOH removed | | | |
|---|---|---|---|---|---|---|
| Water/EtOH: | | water after | after mixing | | % of total | EtOH in |
| Absorbent | EtOH in | mixing with | with Absorbent | EtOH extracted | ethanol | Absorbent |
| Ratio | water (%) | Absorbent (%) | (%) | from H₂O (ml) | removed | (%) |
| 1:1 | 4.924 | 4.599 | 0.325 | 0.0065 | 6.60% | 0.497 |
| 1:5 | 4.924 | 3.329 | 1.595 | 0.01595 | 32.39% | 0.362 |
| 1:10 | 4.924 | 2.443 | 2.481 | 0.02481 | 50.39% | 0.267 |
| 1:20 | 4.924 | 1.61 | 3.314 | 0.03314 | 67.30% | 0.181 |
| 1:40 | 4.924 | 0.943 | 3.981 | 0.03981 | 80.85% | 0.099 |

TABLE 9(b)
Ethanol recovery with different volume of Water/Ethanol to Absorbent ratio

| | | | | | | Volumetric | |
|---|---|---|---|---|---|---|---|
| Water/EtOH: | EtOH | EtOH in | H₂O in | H₂O in | H₂O | ratio of | |
| Absorbent | recovery | Absorbent | Absorbent | Absorbent | crossover | H₂O/EtOH | |
| Ratio | (%) | (ml) | (%) | (ml) | (%) | in Absorbent | K_{aw} |
| 1:1 | 10.09% | 0.00497 | 0.372 | 0.00372 | 0.39% | 0.748 | 0.108067 |
| 1:5 | 36.76% | 0.0181 | 0.366 | 0.0183 | 1.92% | 1.011 | 0.108741 |
| 1:10 | 54.22% | 0.0267 | 0.353 | 0.0353 | 3.71% | 1.322 | 0.109292 |
| 1:20 | 73.52% | 0.0362 | 0.347 | 0.0694 | 7.30% | 1.917 | 0.112422 |
| 1:40 | 80.42% | 0.0396 | 0.345 | 0.138 | 14.51% | 3.485 | 0.104984 |

It can be seen from the results tabulated and from FIG. 8 that with the increase in the volume of Absorbent relative to the volume of the ethanol to water mixture, more ethanol was extracted from the water.

It can be further seen from FIG. 9, that the relative amounts of water in the Absorbent increases as the ratio of the Absorbent increases. It can also be seen, for example that the equilibrium constant K_{aw} at the water and ethanol:absorbent ratio of 1:20 is 0.112422.

### Example 8: Tripentylamine (TPA): 2-octanone Alcohol Recovery at a 40:1 Ratio of Absorbent to Water/Ethanol Solution

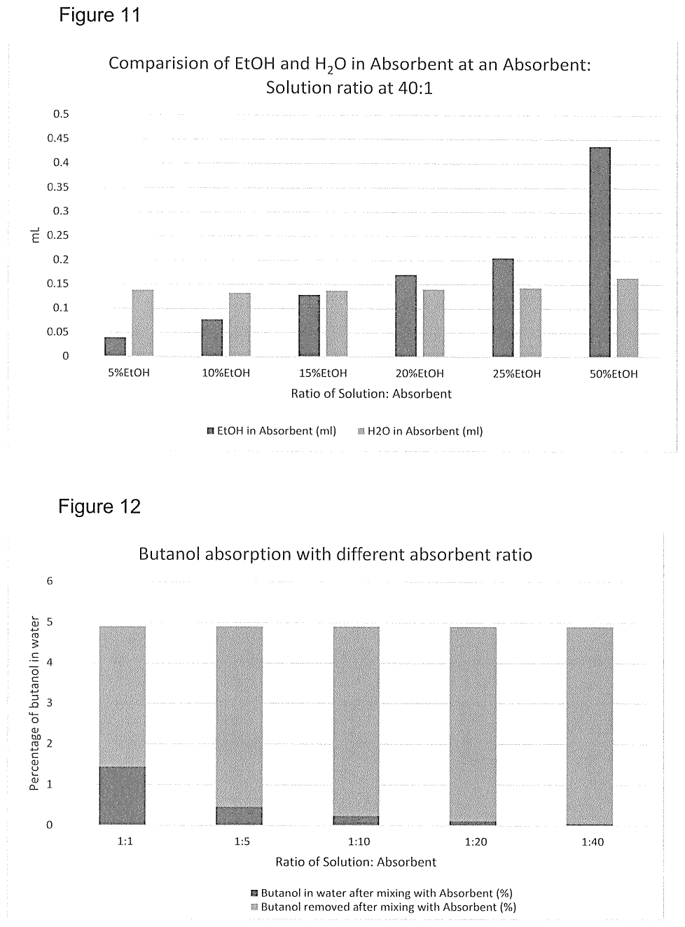

1 ml samples of ethanol/water solution at varying percentages of ethanol (5% ethanol, 10% ethanol, 15% ethanol, 20% ethanol, 25% ethanol, 50% ethanol) was added to 40 ml of the alcohol recovery solution/Absorbent (Tripentylamine and 2-octanone). The resulting mixture was vortexed, then centrifuged to separate. The Absorbent (top phase) and the mixture (bottom phase) were analysed by GC and the alcohol and water crossover was calculated using the equation outline in Example 7 above. The results are tabulated in Tables 10(a) and 10(b) below and results shown in FIGS. 10 and 11.

TABLE 10(a)
Ethanol recovery and water crossover at an Absorbent ratio of 40:1

| | | EtOH in | EtOH removed | | | |
|---|---|---|---|---|---|---|
| | | water after | after mixing | | % of total | EtOH in |
| | EtOH in | mixing with | with Absorbent | EtOH extracted | EtOH | Absorbent |
| Sample | water (%) | Absorbent (%) | (%) | from H₂O (ml) | removed | (%) |
| 5% EtOH | 4.924 | 0.943 | 3.981 | 0.03981 | 80.85% | 0.099 |
| 10% EtOH | 9.544 | 1.928 | 7.616 | 0.07616 | 79.80% | 0.193 |
| 15% EtOH | 15.732 | 3.002 | 12.73 | 0.1273 | 80.92% | 0.32 |
| 20% EtOH | 20.855 | 4.178 | 16.677 | 0.16677 | 79.97% | 0.425 |
| 25% EtOH | 25.857 | 4.852 | 21.005 | 0.21005 | 81.24% | 0.513 |
| 50% EtOH | 52.18 | 9.439 | 42.741 | 0.42741 | 81.91% | 1.09 |

TABLE 10(b)
Ethanol recovery and water crossover at an Absorbent ratio of 40:1

| | EtOH recovery | | | | Volumetric | |
|---|---|---|---|---|---|---|
| | based on | H₂O in | H₂O in | H₂O | ratio of | |
| | Absorbent | Absorbent | Absorbent | crossover | H₂O/EtOH | |
| Sample | layer (%) | (%) | (ml) | (%) | in Absorbent | K_{aw} |
| 5% EtOH | 80.42% | 0.345 | 0.138 | 14.51% | 3.484848 | 0.104984 |
| 10% EtOH | 80.89% | 0.331 | 0.1324 | 14.64% | 1.715026 | 0.100104 |
| 15% EtOH | 81.36% | 0.342 | 0.1368 | 16.23% | 1.06875 | 0.106596 |
| 20% EtOH | 81.52% | 0.349 | 0.1396 | 17.64% | 0.821176 | 0.101723 |
| 25% EtOH | 79.36% | 0.357 | 0.1428 | 19.26% | 0.695906 | 0.10573 |
| 50% EtOH | 83.56% | 0.409 | 0.1636 | 34.21% | 0.375229 | 0.115478 |

It can be seen that the ethanol recovery is around 80% even with varying concentrations of ethanol in the ethanol/water sample. It is to be noted, however, that with an increase in the ethanol concentration the water crossover increases as well and this can be seen in FIG. 10.

### Example 9: Tripentylamine (TPA): 2-Octanone (0.5:1 Molar Ratio) Used for Butan-1-ol Recovery

A 5% butan-1-ol (2 mls) in water (in 38 mls of water) solution was prepared and analysed by GC.

The alcohol recovery solution (also referred to as the alcohol absorbent solution) was prepared as a 0.5:1 mole ratio of tripentylamine and 2-octanone by combining 289 ml of tripentylamine to 311 ml of 2-octanone and mixing by shaking for 10 secs.

Gas chromatography calibration curves of butan-1-ol with water and tripentylamine, 2-octanone with water and tripentylamine, 2-octanone and butan-1-ol were obtained by using the GC parameters described above in Example 6.

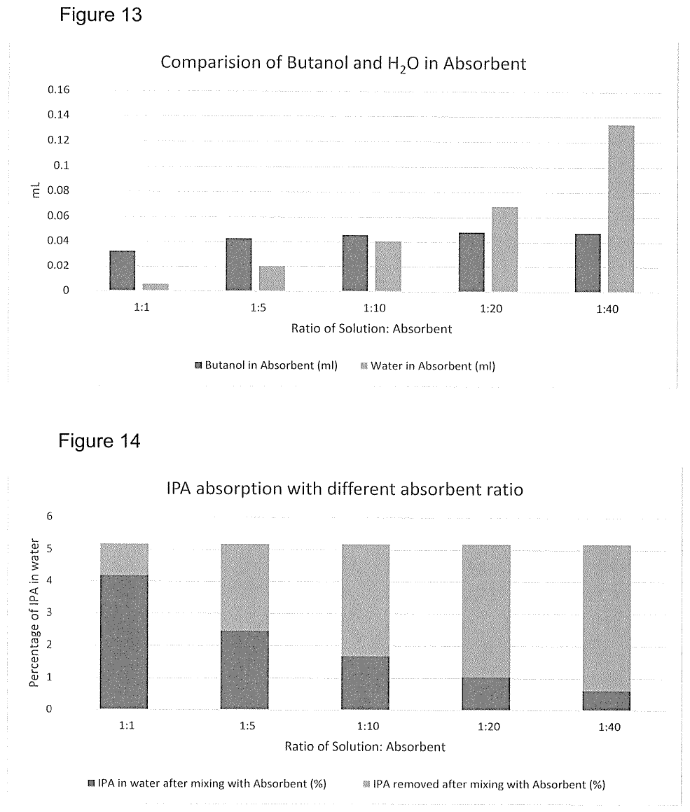

1 ml of the 5% butan-1-ol solution was added to various ratios (1:1, 1:5, 1:10, 1:20 and 1:40) of the tripentylamine and 2-octanone alcohol recovery solution as detailed above in Example 7. The resulting mixture was vortexed for 30 seconds and then centrifuged for 1 min at 4000 rpm to separate into two layers. The alcohol recovery solution of tripentylamine and 2-octanone (or alcohol absorbent solution “Absorbent”) was the top phase and the bottom phase was the alcohol solution in water. The composition of the top phase and the bottom phase were analysed by GC to determine a number of parameters, including the percentage of ethanol in water after mixing with the Absorbent, the butan-1-ol remaining in the water after mixing with the Absorbent. These parameters were then used to calculate the butan-1-ol extracted from the water, the percentage of the total butan-1-ol removed, the mls and percentage of butan-1-ol in the Absorbent, the mls and percentage of water in the Absorbent and the percentage of water crossover into the Absorbent. The butan-1-ol alcohol recovery and water crossover was calculated using the equations below and the results are shown in Tables 11(a) and 11(b) and in FIGS. 12 and 13.

TABLE 11(a)
Butan-1-ol recovery with different volume of Water/Butan-1-ol to Absorbent ratio

| | | Butan-1-ol | Butan-1-ol | | | |
|---|---|---|---|---|---|---|
| | | in water | removed | Butan-1-ol | | |
| Water: | Butan-1-ol | after mixing | after mixing | extracted | % of total | |
| Absorbent | in water | with Absorbent | with Absorbent | from H₂O | Butan-1-ol | Butan-1-ol in |
| Ratio | (%) | (%) | (%) | (ml) | removed | Absorbent (%) |
| 1:1 | 4.903 | 1.436 | 3.467 | 0.03467 | 70.71% | 3.214 |
| 1:5 | 4.903 | 0.46 | 4.443 | 0.04443 | 90.62% | 0.853 |
| 1:10 | 4.903 | 0.24 | 4.663 | 0.04663 | 95.11% | 0.456 |
| 1:20 | 4.903 | 0.113 | 4.79 | 0.0479 | 97.70% | 0.24 |
| 1:40 | 4.903 | 0.045 | 4.858 | 0.04858 | 99.08% | 0.119 |

TABLE 11(b)
Butan-1-ol recovery with different volume of Water/Butan-1-ol to Absorbent ratio

| | | | | | | Volumetric | |
|---|---|---|---|---|---|---|---|
| Water: | Butan-1-ol | Butan-1-ol | Water in | Water in | Water | ratio of | |
| Absorbent | recovery | in Absorbent | Absorbent | Absorbent | crossover | H₂O/Butan-1-ol | |
| Ratio | (%) | (ml) | (%) | (ml) | (%) | in Absorbent | K_{aw} |
| 1:1 | 65.55% | 0.03214 | 0.567 | 0.00567 | 0.60% | 0.18 | 2.24 |
| 1:5 | 86.99% | 0.04265 | 0.409 | 0.02045 | 2.15% | 0.48 | 1.85 |
| 1:10 | 93.00% | 0.0456 | 0.406 | 0.0406 | 4.27% | 0.89 | 1.90 |
| 1:20 | 97.90% | 0.048 | 0.343 | 0.0686 | 7.21% | 1.43 | 2.12 |
| 1:40 | 97.08% | 0.0476 | 0.335 | 0.134 | 14.09% | 2.82 | 2.64 |

It can be seen from the results tabulated and from FIG. 12 that with the increase in the volume of Absorbent relative to the volume of the butan-1-ol to water mixture, more butan-1-ol was extracted from the water.

It can be further seen from FIG. 13, that the relative amounts of water in the Absorbent increases as the ratio of the Absorbent increases. It can also be seen, for example that the equilibrium constant K_{aw} at the water and butan-1-ol:absorbent ratio of 1:20 is 2.12.

### Example 10: Tripentylamine (TPA): 2-Octanone (0.5:1 Molar Ratio) Used for Isopropyl Alcohol Recovery

A 5% isopropyl alcohol (2 mls) in water (in 38 mls of water) solution was prepared and analysed by GC. The GC parameters used are detailed below:

IPA GC Parameters

Method of Analysing IPA in Absorbent

| Parameter | Setting | |
|---|---|---|
| Injection Volume | 0.5 | μL |
| Injection temperature | 250° | C. |
| Injection mode | Split | |
| Split ratio | 50.0 | |
| Carrier gas | He | |
| Carrier gas pressure | 53.1 | kPa |
| Column | SH-Rxi-624Sil MS | |
| Column flow | 1.16 | mL/min |
| Liner velocity | 24.0 | cm/s |
| Column length | 30.0 | m |
| Column inner diameter | 0.32 | |
| Column method | Gradient | |
| Column temperature | 250.0° | C. |
| Total time | 16 | min |
| Detector | TCD | |
| TCD sample rate | 40 | ms |
| TCD current | 60 | mA |
| Makeup gas | He | |
| Makeup flow | 8.0 | mL/min |
| TCD temperature | 200° | C. |

Column Method:

| Rate (° C./min) | Temperature (° C.) | Hold Time (min) |
|---|---|---|
| | 100.00 | 2.00 |
| 15.0 | 250 | 4.00 |

Total program time 16.00 min

Method of Analysing IPA in Water

| Parameter | Setting | |
|---|---|---|
| Injection Volume | 0.5 | μL |
| Injection temperature | 250° | C. |
| Injection mode | Split | |
| Split ratio | 50.0 | |
| Carrier gas | He | |
| Carrier gas pressure | 53.1 | kPa |
| Column | SH-Rxi-624Sil MS | |
| Column flow | 1.16 | mL/min |
| Liner velocity | 24.0 | cm/s |
| Column length | 30.0 | m |
| Column inner diameter | 0.32 | |
| Column method | Gradient | |
| Column temperature | 125.0° | C. |
| Total time | 16 | min |
| Detector | TCD | |
| TCD sample rate | 40 | ms |
| TCD current | 60 | mA |
| Makeup gas | He | |
| Makeup flow | 8.0 | mL/min |
| TCD temperature | 200° | C. |

Column Method:

| Rate (° C./min) | Temperature (° C.) | Hold Time (min) |
|---|---|---|
| | 60.00 | 4.00 |
| 10.0 | 125 | 1.00 |

Total program time 11.50 min

The alcohol recovery solution (also referred to as the alcohol absorbent solution) was prepared as a 0.5:1 mole ratio of tripentylamine and 2-octanone by combining 289 ml of tripentylamine to 311 ml of 2-octanone and mixing by shaking for 10 secs.

Gas chromatography calibration curves of isopropyl alcohol with water and tripentylamine, 2-octanone with water and tripentylamine, 2-octanone and isopropyl alcohol were obtained by GC using the parameters detailed above in this example.

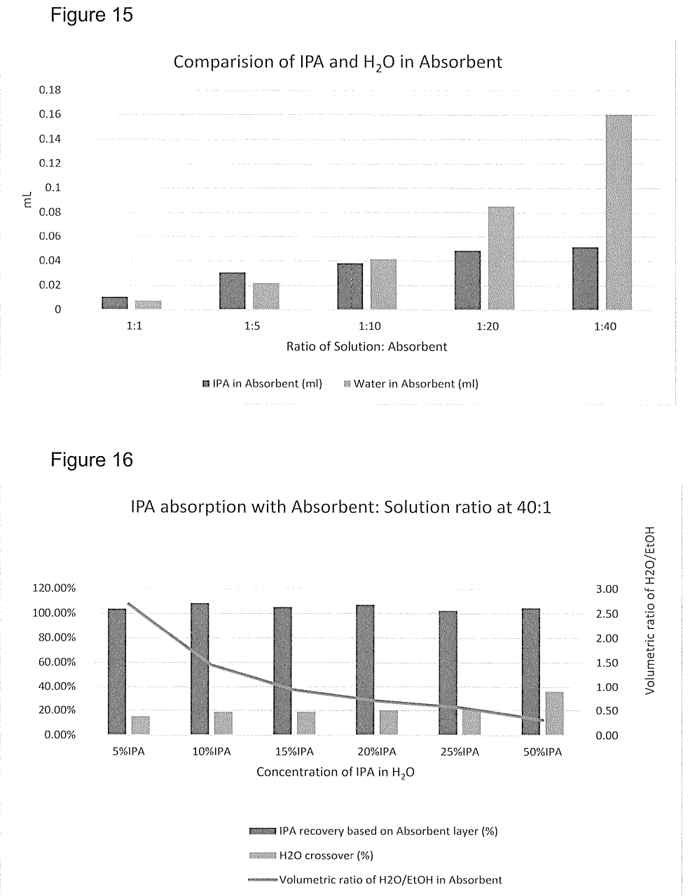

1 ml of the 5% isopropyl alcohol solution was added to various ratios (1:1, 1:5, 1:10, 1:20 and 1:40) of the tripentylamine and 2-octanone alcohol recovery solution as detailed in Example 7 above. The resulting mixture was vortexed for 30 seconds and then centrifuged for 1 min at 4000 rpm to separate into two layers. The alcohol recovery solution of tripentylamine and 2-octanone (or alcohol absorbent solution “Absorbent”) was the top phase and the bottom phase was the alcohol solution in water. The composition of the top phase and the bottom phase were analysed by GC to determine a number of parameters, including the percentage of isopropyl alcohol in water after mixing with the Absorbent, the isopropyl alcohol remaining in the water after mixing with the Absorbent. These parameters were then used to calculate the isopropanol extracted from the water, the percentage of the total isopropyl alcohol removed, the mls and percentage of isopropyl alcohol in the Absorbent, the mls and percentage of water in the Absorbent and the percentage of water crossover into the Absorbent. The isopropyl alcohol recovery and water crossover was calculated using the equations outlined in Example 7 above and the results are shown in Tables 12(a) and 12(b) and in FIGS. 14 and 15.

TABLE 12(a)
Isopropyl alcohol recovery with different volume of Water/isopropyl alcohol to Absorbent ratio

| | | IPA in water | IPA removed | | | |
|---|---|---|---|---|---|---|
| Water: | | after mixing | after mixing | | | |
| Absorbent | IPA in | with Absorbent | with Absorbent | IPA extracted | % of total | IPA in |
| Ratio | water (%) | (%) | (%) | from H₂O (ml) | IPA removed | Absorbent (%) |
| 1:1 | 5.358 | 4.214 | 1.144 | 0.01144 | 21.35 | 1.066 |
| 1:5 | 5.358 | 2.612 | 2.746 | 0.02746 | 51.25 | 0.61 |
| 1:10 | 5.358 | 1.794 | 3.564 | 0.03564 | 66.52 | 0.38 |
| 1:20 | 5.358 | 1.15 | 4.208 | 0.04208 | 78.54 | 0.243 |
| 1:40 | 5.358 | 0.575 | 4.783 | 0.04783 | 89.27 | 0.129 |

TABLE 12(b)
Isopropyl alcohol recovery with different volume
of Water/isopropyl alcohol to Absorbent ratio

| | | | | | | Volumetric | |
|---|---|---|---|---|---|---|---|
| Water: | IPA | IPA in | Water in | Water in | Water | ratio of | |
| Absorbent | recovery | Absorbent | Absorbent | Absorbent | crossover | H2O/IPA | |
| Ratio | (%) | (ml) | (%) | (ml) | (%) | in Absorbent | K_{aw} |
| 1:1 | 19.90% | 0.01066 | 0.746 | 0.00746 | 0.79% | 0.70 | 0.2529 |
| 1:5 | 56.92% | 0.0305 | 0.438 | 0.0219 | 2.31% | 0.72 | 0.2335 |
| 1:10 | 70.92% | 0.038 | 0.415 | 0.0415 | 4.38% | 1.09 | 0.2118 |
| 1:20 | 90.71% | 0.0486 | 0.426 | 0.0852 | 9.00% | 1.75 | 0.2113 |
| 1:40 | 96.30% | 0.0516 | 0.401 | 0.1604 | 16.95% | 3.11 | 0.2243 |

It can be seen from the results tabulated and from FIG. 14 that with the increase in the volume of Absorbent relative to the volume of the isopropyl alcohol to water mixture, more isopropyl alcohol was extracted from the water.

It can be further seen from FIG. 15, that the relative amounts of water in the Absorbent increases as the ratio of the Absorbent increases. It can also be seen, for example that the equilibrium constant K_{aw} at the water and isopropyl:absorbent ratio of 1:20 is 2.113.

### Example 11: Tripentylamine (TPA): 2-octanone Alcohol Recovery at a 40:1 Ratio of Absorbent to Water/Isopropyl Alcohol Solution

1 ml samples of isopropyl alcohol/water solution at varying percentages of ethanol (5% isopropyl alcohol I, 10% isopropyl alcohol, 15% isopropyl alcohol, 20% isopropyl alcohol, 25% isopropyl alcohol, 50% isopropyl alcohol I) was added to 40 ml of the alcohol recovery solution/Absorbent (tripentylamine and 2-octanone). The resulting mixture was vortexed, then centrifuged to separate. The Absorbent (top phase) and the mixture (bottom phase) were analysed by GC and the alcohol and water crossover was calculated using the equation outlined in Example 7 above. The results are tabulated in Tables 13(a) and 13(b) below and results shown in FIG. 16.

TABLE 13(a)
Isopropyl recovery and water crossover at an Absorbent ratio of 40:1

| | | IPA in water | IPA removed | | | |
|---|---|---|---|---|---|---|
| | | after mixing | after mixing | | IPA recovery | |
| | IPA in | with Absorbent | with Absorbent | IPA extracted | based on water | IPA in |
| Sample | water (%) | (%) | (%) | from H2O (ml) | layer (%) | Absorbent (%) |
| 5% IPA | 5.175 | 0.608 | 4.567 | 0.04567 | 88.25% | 0.134 |
| 10% IPA | 10.7 | 1.164 | 9.536 | 0.09536 | 89.12% | 0.289 |
| 15% IPA | 16.35 | 1.696 | 14.654 | 0.14654 | 89.63% | 0.429 |
| 20% IPA | 21.199 | 2.233 | 18.966 | 0.18966 | 89.47% | 0.566 |
| 25% IPA | 27.129 | 2.724 | 24.405 | 0.24405 | 89.96% | 0.693 |
| 50% IPA | 53.427 | 5.321 | 48.106 | 0.48106 | 90.04% | 1.395 |

TABLE 13(b)
Isopropyl recovery and water crossover at an Absorbent ratio of 40:1

| | IPA recovery | | | | | Volumetric |
|---|---|---|---|---|---|---|
| | based on | IPA in | H₂O in | H₂O in | H₂O | ratio of |
| | Absorbent | Absorbent | Absorbent | Absorbent | crossover | H₂O/IPA |
| Sample | layer (%) | (ml) | (%) | (ml) | (%) | in Absorbent |
| 5% IPA | 103.57% | 0.0536 | 0.359 | 0.1436 | 15.14% | 2.68 |
| 10% IPA | 108.04% | 0.1156 | 0.417 | 0.1668 | 18.68% | 1.44 |
| 15% IPA | 104.95% | 0.1716 | 0.398 | 0.1592 | 19.03% | 0.93 |
| 20% IPA | 106.80% | 0.2264 | 0.394 | 0.1576 | 20.00% | 0.70 |
| 25% IPA | 102.18% | 0.2772 | 0.387 | 0.1548 | 21.24% | 0.56 |
| 50% IPA | 104.44% | 0.558 | 0.421 | 0.1684 | 36.16% | 0.30 |

It can be seen that the isopropyl recovery is around 90% even with varying concentrations of isopropyl alcohol in the isopropyl/water sample. It is to be noted, however, that with an increase in the isopropyl alcohol concentration the water crossover increases as well and this can be seen in FIG. 16.

### Example 12: Tripentylamine (TPA): 2-Octanone (0.5:1 Molar Ratio) Used for Methanol Recovery

A 5% methanol (2 mls) in water (in 38 mls of water) solution was prepared and analysed by GC.

The alcohol recovery solution (also referred to as the alcohol absorbent solution) was prepared as a 0.5:1 mole ratio of tripentylamine and 2-octanone by combining 289 ml of tripentylamine to 311 ml of 2-octanone and mixing by shaking for 10 secs.

Gas chromatography calibration curves of methanol with water and tripentylamine, 2-octanone with water and tripentylamine, 2-octanone and isopropyl alcohol were obtained by GC per the parameters detailed in this Example.

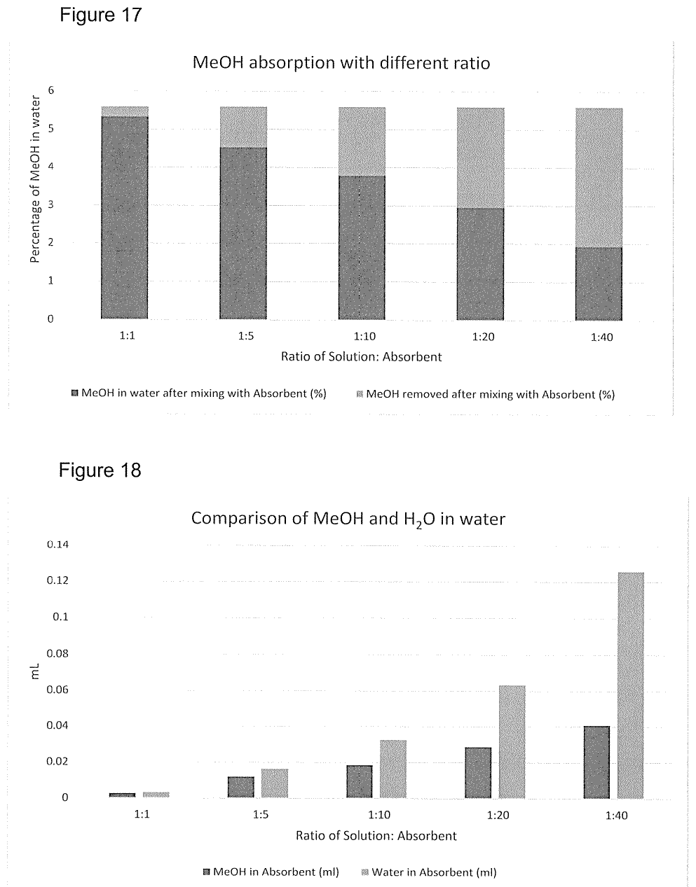

1 ml of the 5% methanol solution was added to various ratios (1:1, 1:5, 1:10, 1:20 and 1:40) of the tripentylamine and 2-octanone alcohol recovery solution were prepared as described above in Example 7. The resulting mixture was vortexed for 30 seconds and then centrifuged for 1 min at 4000 rpm to separate into two layers. The alcohol recovery solution of tripentylamine and 2-octanone (or alcohol absorbent solution “Absorbent”) was the top phase and the bottom phase was the alcohol solution in water. The composition of the top phase and the bottom phase were analysed by GC per the parameters detailed in Example 6 to determine a number of parameters, including the percentage of methanol in water after mixing with the Absorbent, the methanol remaining in the water after mixing with the Absorbent. These parameters were then used to calculate the methanol extracted from the water, the percentage of the total isopropyl alcohol removed, the mls and percentage of methanol in the Absorbent, the mls and percentage of water in the Absorbent and the percentage of water crossover into the Absorbent. The methanol recovery and water crossover was calculated using the equations outlined in Example 7 and the results are shown in Tables 14(a) and 14(b) and in FIGS. 17 and 18.

TABLE 14(a)
Methanol recovery with different volume of Water/MeOH alcohol to Absorbent ratio

| | | MeOH in water | MeOH removed | | | |
|---|---|---|---|---|---|---|
| Water: | | after mixing | after mixing | | | |
| Absorbent | MeOH in | with Absorbent | with Absorbent | MeOH extracted | % of total | MeOH in |
| Ratio | water (%) | (%) | (%) | from H₂O (ml) | MeOH removed | Absorbent (%) |
| 1:1 | 5.583 | 5.326 | 0.257 | 0.00257 | 4.60 | 0.261 |
| 1:5 | 5.583 | 4.529 | 1.054 | 0.01054 | 18.88 | 0.238 |
| 1:10 | 5.583 | 3.774 | 1.809 | 0.01809 | 32.40 | 0.184 |
| 1:20 | 5.583 | 2.952 | 2.631 | 0.02631 | 47.13 | 0.143 |
| 1:40 | 5.583 | 1.927 | 3.656 | 0.03656 | 65.48 | 0.102 |

TABLE 14(b)
Methanol recovery with different volume of Water/MeOH alcohol to Absorbent ratio

| | | | | | | Volumetric | |
|---|---|---|---|---|---|---|---|
| Water: | MeOH | MeOH in | Water in | Water in | Water | ratio of | |
| Absorbent | recovery | Absorbent | Absorbent | Absorbent | crossover | H2O/MeOH | |
| Ratio | (%) | (ml) | (%) | (ml) | (%) | in Absorbent | K_{aw} |
| 1:1 | 4.67% | 0.00261 | 0.314 | 0.00314 | 0.33% | 1.20 | 0.049005 |
| 1:5 | 21.31% | 0.0119 | 0.322 | 0.0161 | 1.71% | 1.35 | 0.05255 |
| 1:10 | 32.96% | 0.0184 | 0.324 | 0.0324 | 3.43% | 1.76 | 0.048755 |
| 1:20 | 51.23% | 0.0286 | 0.317 | 0.0634 | 6.71% | 2.22 | 0.048442 |
| 1:40 | 73.08% | 0.0408 | 0.314 | 0.1256 | 13.30% | 3.08 | 0.052932 |

It can be seen from the results tabulated and from FIG. 17 that with the increase in the volume of Absorbent relative to the volume of the methanol to water mixture, more methanol was extracted from the water.

It can be further seen from FIG. 18, that the relative amounts of water in the Absorbent increases as the ratio of the Absorbent increases. It can also be seen, for example that the equilibrium constant K_{aw} at the water and methanol:absorbent ratio of 1:20 is 20.0484.

### Example 13: Tripentylamine (TPA): 2-Octanone (0.5:1 Molar Ratio) Used for Tert-Butanol Recovery

A 5% tert-butanol (2 mls) in water (in 38 mls of water) solution was prepared and analysed by GC using the same GC parameters as described above for Example 10.

The alcohol recovery solution (also referred to as the alcohol absorbent solution) was prepared as a 0.5:1 mole ratio of tripentylamine and 2-octanone by combining 289 ml of tripentylamine to 311 ml of 2-octanone and mixing by shaking for 10 secs.

Gas chromatography calibration curves of tert-butanol with water and tripentylamine, 2-octanone with water and tripentylamine, 2-octanone and tert-butanol were obtained by GC using the parameters detailed above in Example 10.

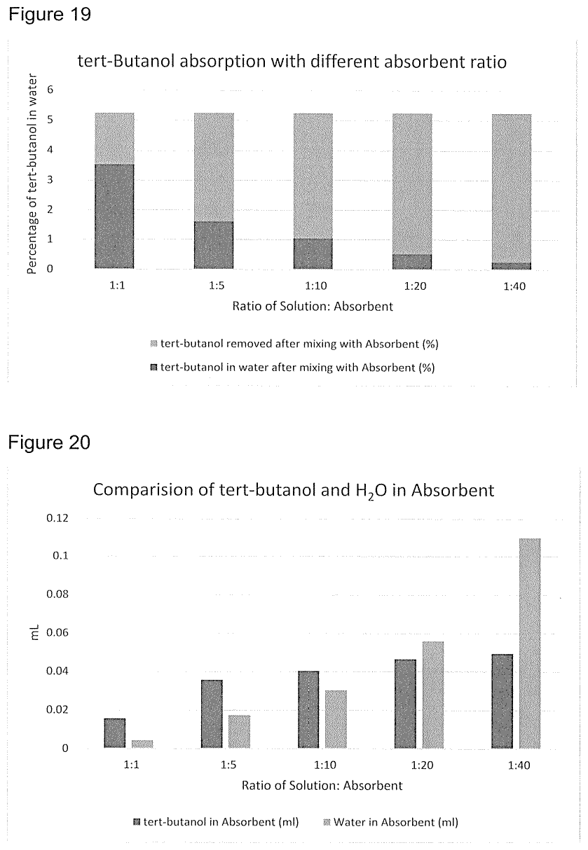

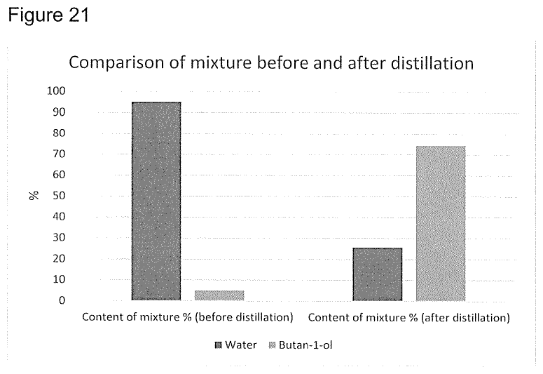

1 ml of the 5% isopropyl alcohol solution was added to various ratios (1:1, 1:5, 1:10, 1:20 and 1:40) of the tripentylamine and 2-octanone alcohol recovery solution as detailed in Example 7 above. The resulting mixture was vortexed for 30 seconds and then centrifuged for 1 min at 4000 rpm to separate into two layers. The alcohol recovery solution of tripentylamine and 2-octanone (or alcohol absorbent solution “Absorbent”) was the top phase and the bottom phase was the alcohol solution in water. The composition of the top phase and the bottom phase were analysed by GC to determine a number of parameters, including the percentage of tert-butanol in water after mixing with the Absorbent, the tert-butanol remaining in the water after mixing with the Absorbent. These parameters were then used to calculate the tert-butanol extracted from the water, the percentage of the total tert-butanol removed, the mls and percentage of tert-butanol in the Absorbent, the mls and percentage of water in the Absorbent and the percentage of water crossover into the Absorbent. The tert-butanol recovery and water crossover was calculated using the equations outlined in Example 7 above and the results are shown in Tables 15(a) and 15(b) and in FIGS. 19 and 20.

TABLE 15(a)
Tert-Butanol recovery and water crossover at an Absorbent ratio of 40:1

| | | tert-butanol in | tert-butanol | tert-butanol | | |
|---|---|---|---|---|---|---|
| Water: | | water after | removed after | extracted | % of total | tert-butanol |
| Absorbent | tert-butanol | mixing with | mixing with | from H2O | tert-butanol | in Absorbent |
| Ratio | in water (%) | Absorbent (%) | Absorbent (%) | (ml) | removed | (%) |
| 1:1 | 5.237 | 3.534 | 1.703 | 0.01703 | 32.52% | 1.566 |
| 1:5 | 5.237 | 1.614 | 3.623 | 0.03623 | 69.18% | 0.716 |
| 1:10 | 5.237 | 1.038 | 4.199 | 0.04199 | 80.18% | 0.404 |
| 1:20 | 5.237 | 0.524 | 4.713 | 0.04713 | 89.99% | 0.232 |
| 1:40 | 5.237 | 0.251 | 4.986 | 0.04986 | 95.21% | 0.123 |

TABLE 15(b)
Tert-Butanol recovery and water crossover at an Absorbent ratio of 40:1

| | | | | | | Volumetric | |
|---|---|---|---|---|---|---|---|
| Water: | | tert-butanol | Water in | Water in | Water | ratio of | |
| Absorbent | tert-butanol | in Absorbent | Absorbent | Absorbent | crossover | H₂O/tert-Butanol | |
| Ratio | recovery (%) | (ml) | (%) | (ml) | (%) | in Absorbent | K_{aw} |
| 1:1 | 29.90% | 0.01566 | 0.431 | 0.00431 | 0.45% | 0.28 | 0.4431 |
| 1:5 | 68.36% | 0.0358 | 0.346 | 0.0173 | 1.83% | 0.48 | 0.4436 |
| 1:10 | 77.14% | 0.0404 | 0.305 | 0.0305 | 3.22% | 0.75 | 0.3892 |
| 1:20 | 88.60% | 0.0464 | 0.279 | 0.0558 | 5.89% | 1.20 | 0.4427 |
| 1:40 | 93.95% | 0.0492 | 0.275 | 0.11 | 11.61% | 2.24 | 0.4900 |

It can be seen from the results tabulated and from FIG. 19 that with the increase in the volume of Absorbent relative to the volume of the tert-butanol to water mixture, more tert-butanol alcohol was extracted from the water.

It can be further seen from FIG. 19, that the relative amounts of water in the Absorbent increases as the ratio of the Absorbent increases. It can also be seen, for example that the equilibrium constant K_{aw} at the water and isopropyl:absorbent ratio of 1:20 is 0.4427.

### Example 14: An Alcohol Recovery Distillation

One of the butanol water solutions (6.5 g) recovered from Example 9 above was distilled at 40 degrees Celsius (inner temperature) and at −650 mmHg. The mixture was analysed by GC and the results shown in Table 16 and FIG. 19.

TABLE 16

| | Content of mixture % | Content of mixture % |
|---|---|---|
| Component | (before distillation) | (after distillation) |
| Water | 95.051 | 25.64 |
| Butan-1-ol | 4.949 | 74.36 |

It is to be appreciated from the results provided that it is possible to concentrate the alcohol from feed stream containing the alcohol and then remove by a means, such as by a distillation for example, to purify and isolate the alcohol.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention.

Accordingly, one of ordinary skill in the art will readily appreciate from the disclosure that later modifications, substitutions, and/or variations performing substantially the same function or achieving substantially the same result as embodiments described herein may be utilised according to such related embodiments of the present invention. Thus, the invention is intended to encompass, within its scope, the modifications, substitutions, and variations to processes, manufactures, compositions of matter, compounds, means, methods, and/or steps disclosed herein.

1. A composition for use in recovering an alcohol from an aqueous solution the composition comprising:
a) a recovery solution comprising at least one tertiary amine containing compound and at least one enolisable carbonyl; and,
b) an aqueous process solution comprising the alcohol,
wherein the recovery solution is not miscible with the aqueous process solution and at least a portion of the alcohol migrates from the aqueous process solution in the absence of a semipermeable membrane into the recovery solution.

2. A composition for use in recovering an alcohol from an aqueous solution the composition comprising:
a) a recovery solution comprising at least one tertiary amine containing compound and at least one enolisable carbonyl; and,
b) an aqueous process solution comprising the alcohol,
wherein the recovery solution and the aqueous process solution are in direct contact, not miscible and at least a portion of the alcohol migrates from the aqueous process solution into the recovery solution.

3. A composition for use in recovering alcohol from an aqueous solution the composition comprising:
a) a recovery solution comprising at least one tertiary amine containing compound and at least one enolisable carbonyl; and,
b) an aqueous process solution comprising the alcohol,
wherein the recovery solution is not miscible with the aqueous solution.

4. The composition as claimed in claim 1, claim 2 or claim 3, wherein the at least one enolisable carbonyl is of Formula I, 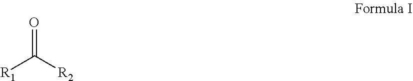
wherein
a) R₁ and R₂ are independently selected from a —C₁-C₂₀ alkyl or a —C₃-C₇ monocyclic; or
b) one of R₁ or R₂ is selected from a —O—(C₁-C₇ alkyl) and the other is selected from a —C₁-C₇ alkyl, or
c) R₁ and R₂, together with the carbonyl of Formula I, form a 3-15 membered monocyclic ketone or a 3-15 membered monocyclic heterocyclic ketone or acetophenone.

5. The composition as claimed in claim 4, wherein the recovery solution includes a combination of more than one enolisable carbonyl of Formula I.

6. The composition as claimed in any one of claims 1 to 5, wherein the recovery solution includes a combination of more than one tertiary amine containing compound.

7. The composition as claimed in any one of claim 1 to 6, wherein the at least one tertiary amine containing compound is selected from a conjugated, aliphatic, asymmetric or cyclic tertiary amine.

8. The composition as claimed in claim 7, wherein the at least one tertiary amine containing compound is selected from one or more of the following:

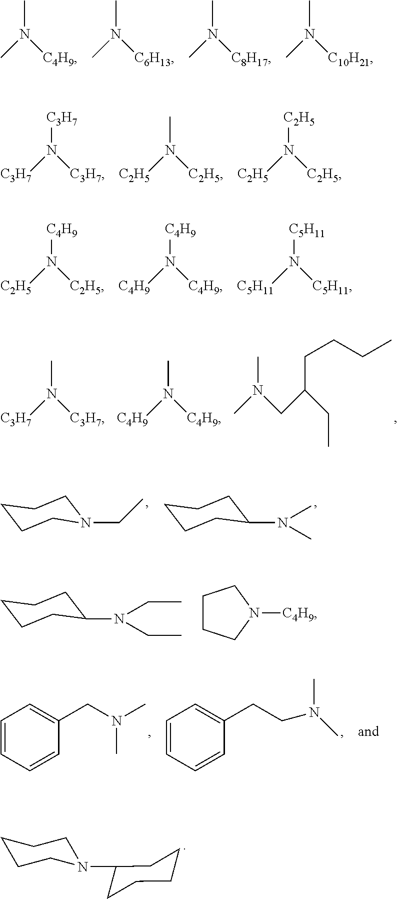

9. The composition as claimed in claim 7, wherein the at least one tertiary amine containing compound is selected from a —N(C₁-C₇ alkyl)₃.

10. The composition as claimed in claim 9, wherein the at least one tertiary amine containing compound is —N(C₅ alkyl)₃ (tripentylamine) or —N(C₄alkyl)₃ tributylamine.

11. The composition as claimed in any one of claims 4 to 10, wherein R₁ and R₂ of Formula I are independently selected from a —C₁-C₂₀ alkyl.

12. The composition as claimed in claim 11, wherein the enolisable carbonyl of Formula I is 5-nonanone.

13. The composition as claimed in any one of claims 4 to 12, wherein the one or more enolisable carbonyls of Formula I is 2-octanone.

14. The composition as claimed in any one of claims 4 to 12, wherein each R₁ and R₂ are further substituted with one or more substituents selected from -halo, —OH, —CN, —NO₂, —C≡CH, —SH, —C₁-C₇ alkyl, —(C₁-C₇ alkyl)-OH, —NH₂, —NH(C₁-C₇ alkyl), —N(C₁-C₇ alkyl)₂, —O(C₁-C₇ alkyl), —C(O)—O(—C₁-C₇ alkyl), —C(O)OH; —C(O)—H, or —C(O)—(C₁-C₇ alkyl).

15. The composition as claimed in any one of claims 4 to 14, wherein the molar ratio of the at least one tertiary amine containing compound to the one or more enolisable carbonyls of Formula I are present in a ratio of about 1:99 or 99:1.

16. The composition as claimed in claim 15, wherein the molar ratio of the at least one tertiary amine containing compound to the one or more enolisable carbonyls of Formula I are present in a ratio of about 1:50 or 50:1.

17. The composition as claimed in claim 15 or claim 16, wherein the molar ratio of the at least one tertiary amine containing compound to the enolisable carbonyl of Formula I are present in a ratio of about 1:10 or 10:1.

18. The composition as claimed in any one of claims 15 to 17, wherein the molar ratio of the at least one tertiary amine containing compound to the one or more enolisable carbonyls of Formula I are present in a ratio of about 1:5 or 5:1.

19. The composition as claimed in any one of claims 15 to 18, wherein the molar ratio of the at least one tertiary amine containing compound to the one or more enolisable carbonyls of Formula I are present in a ratio of about 1:3 or 3:1.

20. The composition as claimed in any one of claims 15 to 19, wherein the molar ratio of the at least one tertiary amine containing compound to the one or more enolisable carbonyls of Formula I are present in a ratio of about 1:2 or 2:1.

21. The composition as claimed in any one of claims 15 to 20, wherein the molar ratio of the at least one tertiary amine containing compound to the one or more enolisable carbonyls of Formula I are present in a ratio of about 1:1.

22. The composition as claimed in any one of claims 1 to 21, wherein the alcohol to be recovered is selected from ethanol, butanol, isopropyl alcohol, methanol, tert-butanol or mixtures thereof.

23. The composition as claimed in claim 22, wherein the alcohol is ethanol.

24. A method for separating an alcohol from an aqueous solution using a composition as claimed in any one of claims 1 to 23, the method comprising the steps of:
1) bringing the alcohol containing aqueous solution into contact with the alcohol recovery solution;
2) allowing one or more alcohols in the aqueous solution to at least partially migrate into the solvent recovery solution; and
3) separating the recovered alcohol from the aqueous solution.

25. The method as claimed in claim 24, further including the step of transferring the alcohol recovery solution which is enriched in recovered alcohol to an alcohol regeneration process.

26. The method as claimed in claim 25, wherein the alcohol regeneration process is a distillation process.

27. The method as claimed in claim 26, wherein an entrainer is added to the aqueous solution prior to bringing the aqueous solution into contact with the alcohol recovery solution.

28. The method as claimed in claim 27 wherein the entrainer is a C₁-C₇ alkyl.

29. The method as claimed in claim 27 or claim 28 wherein the entrainer is cyclohexane.

30. The method as claimed in any one of claims 24 to 29, wherein the alcohol to be recovered is selected from ethanol, butanol, isopropyl alcohol, methanol, tert-butanol or mixtures thereof.

31. The method as claimed in claim 30, wherein the alcohol to be recovered is ethanol.

32. The method as claimed in any one of claims 24 to 31, wherein the aqueous solution from which the alcohol is to be recovered is selected from industrial water waste streams, fermentation streams, food & beverage processing effluents, or the like.

33. The method as claimed in claim 32, wherein the aqueous solution from which the alcohol is to be recovered is a fermentation stream.
